(19) United States
(12) Patent Application Publication
(10) Pub. No.: US 2026/0060691 A1
(43) Pub. Date: Mar. 5, 2026

(54) DEVICE FOR RELEASABLY COUPLING AN INNER MEMBER EXTENDING WITHIN AN OUTER MEMBER

(71) Applicant: BOSTON SCIENTIFIC SCIMED, INC., Maple Grove, MN (US)

(72) Inventors: Ajay Gupta, Minneapolis, MN (US); Mark Steven Smith, Coon Rapids, MN (US); Robert M. Wold, Maple Lake, MN (US)

(73) Assignee: BOSTON SCIENTIFIC SCIMED, INC., Maple Grove, MN (US)

(21) Appl. No.: 19/320,380

(22) Filed: Sep. 5, 2025

Related U.S. Application Data
(60) Provisional application No. 63/691,002, filed on Sep. 5, 2024.

(51) Int. Cl. A61B 17/12; A61B 17/00

(52) U.S. Cl. CPC A61B 17/1214; A61B 17/12113; A61B 2017/00477; A61B 2017/12054

(57) ABSTRACT

A medical assembly includes an implantable medical device and an elongate delivery member that is adapted to deliver the implantable medical device. The implantable medical device is releasably secured to the elongate delivery member. An outer sheath is adapted to fit over the elongate delivery member. A removable coupler is adapted to secure the elongate delivery member in position relative to the outer sheath when the removable coupler is disposed between the elongate delivery method and the outer sheath.

### CROSS REFERENCE TO RELATED APPLICATIONS

This application claims the benefit of priority of U.S. Provisional Application No. 63/691,002 filed Sep. 5, 2024, the entire disclosure of which is hereby incorporated by reference.

### TECHNICAL FIELD

The disclosure relates generally to medical devices and more particularly to medical devices that include an inner member extending through an outer member.

### BACKGROUND

A wide variety of intracorporeal medical devices have been developed for medical use, for example, intravascular use. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a medical assembly. The medical assembly includes an implantable medical device and an elongate delivery member that is adapted to deliver the implantable medical device with the implantable medical device releasably secured to the elongate delivery member. An outer sheath is adapted to fit over the elongate delivery member. A removable coupler is adapted to secure the elongate delivery member in position relative to the outer sheath when the removable coupler is disposed between the elongate delivery member and the outer sheath.

Alternatively or additionally, the removable coupler may include a tubular member that extends from an insertion end to a handle end, with an angled cut that extends back from the insertion end.

Alternatively or additionally, the tubular member may have a wall thickness that varies circumferentially about the tubular member.

Alternatively or additionally, the tubular member may have a wall thickness that varies circumferentially from a minimum wall thickness along a first side of the tubular member to a maximum wall thickness along an opposing second side of the tubular member.

Alternatively or additionally, the angled cut may start at the insertion end proximate the first side of the tubular member and may extend back towards the second side of the tubular member.

Alternatively or additionally, the angled cut may extend at an angle ranging from eight degrees to twelve degrees with respect to an outer surface of the tubular member proximal of the angled cut.

Alternatively or additionally, the angled cut may extend at an angle of ten degrees with respect to an outer surface of the tubular member proximal of the angled cut.

Alternatively or additionally, the minimum wall thickness may range from 0.15 millimeters (0.006 inches) to 0.25 millimeters (0.010 inches) and the maximum wall thickness may range from 0.30 millimeters (0.012 inches) to 0.41 millimeters (0.016 inches).

Alternatively or additionally, the minimum wall thickness may be 0.20 millimeters (0.008 inches) and the maximum wall thickness may be 0.36 millimeters (0.014 inches).

Alternatively or additionally, the implantable medical device may include an embolic coil.

Another example may be found in a medical assembly. The medical assembly includes an outer member defining a lumen having an inner diameter (ID) and an inner member having an outer diameter (OD), with the inner member extending within the lumen of the outer member. A removable coupler is adapted to be advanced over the inner member and extend into a space between the ID of the outer member and the OD of the inner member in order to secure the inner member relative to the outer member.

Alternatively or additionally, the removable coupler includes a tubular member extending from an insertion end to a handle end, with an angled cut extending back from the insertion end.

Alternatively or additionally, the tubular member may have a wall thickness that varies circumferentially from a minimum wall thickness along a first side of the tubular member to a maximum wall thickness along an opposing second side of the tubular member.

Alternatively or additionally, the angled cut may start at the insertion end proximate the first side of the tubular member and extends back towards the second side of the tubular member.

Alternatively or additionally, the inner member may include a delivery device for an embolic coil and the outer member may include a sheath extending over the embolic coil.

Another example may be found in a removable coupler that is adapted to releasably secure an inner member to an outer member and that is adapted to be removed prior to use. The removable coupler includes a tubular member that extends from an insertion end to a handle end. The tubular member has a wall thickness that varies circumferentially from a minimum wall thickness along one side of the tubular member to a maximum wall thickness along an opposing side of the tubular member. The tubular member defines a tubular member lumen extending therethrough and that is adapted to accommodate the inner member therein. An angled cut extends back from the insertion end.

Alternatively or additionally, the angled cut may extend at an angle ranging from eight degrees to twelve degrees with respect to an outer surface of the tubular member proximal of the angled cut.

Alternatively or additionally, the minimum wall thickness may range from 0.15 millimeters (0.006 inches) to 0.25 millimeters (0.010 inches) and the maximum wall thickness may range from 0.30 millimeters (0.012 inches) to 0.41 millimeters (0.016 inches).

Alternatively or additionally, the tubular member defines an outer diameter that may range from 1.4 millimeters (0.055 inches) to 1.7 millimeters (0.065 inches) and an inner diameter that may range from 0.90 millimeters (0.035 inches) to 1.0 millimeters (0.040 inches).

Alternatively or additionally, the tubular member may have a uniform composition from the insertion end to the handle end.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of a medical assembly that includes an illustrative removable coupler disposed between an inner member and an outer member;

FIG. 1A is a cross-sectional view taken along the line 1A-1A of FIG. 1;

FIG. 2 is a perspective view of the illustrative removable coupler of FIG. 1;

FIG. 3 is a cross-sectional view taken along line 3-3 of FIG. 2;

FIG. 4 is an enlarged side view of a portion of the illustrative removable coupler of FIG. 2;

FIG. 5 is a graphical representation showing benefits of using a non-concentric tubular member in forming the illustrative removable coupler of FIG. 2;

FIG. 6 is a perspective view of an illustrative delivery wire including several embolic coils coupled to the delivery wire; and

FIG. 7 is a side view showing one of the embolic coils of FIG. 6 in a deployment configuration.

It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

There are many instances in which an inner member or an inner medical device may be used in combination with an outer member or an outer medical device. A variety of different medical devices may be inserted through a guide catheter, for example. In some cases, there may be a desire to maintain the spatial relationship between an inner member and an outer member for a period of time. As an example, an inner member may be used to deliver an implantable medical device while an outer member may be or may include a removable sheath that extends over the implantable medical device. The implantable medical device may be an embolic coil. For example, an embolic coil may be coupled with a delivery wire, and a sheath may extend over the embolic coil and thus, may extend over at least part of the delivery wire. In this example, the delivery wire would be the inner member and the sheath may be the outer member.

In some instances, a medical assembly may include an implantable medical device and an elongate delivery member that is adapted to deliver the implantable medical device. The implantable medical device may be releasably secured to the elongate delivery member. An outer sheath is adapted to fit over the elongate delivery member. The medical assembly includes a removable coupler that is adapted to secure the elongate delivery member in position relative to the outer sheath when the removable coupler is disposed between the elongate delivery member and the outer sheath.

In some cases, the removable coupler may include a tubular member that extends from an insertion end to a handle end. An angled cut may extend back from the insertion end. In some cases, the tubular member may have a wall thickness that varies circumferentially about the tubular member. As an example, the tubular member may have a wall thickness that varies circumferentially from a minimum wall thickness along a first side of the tubular member to a maximum wall thickness along an opposing second side of the tubular member. In some cases, the angled cut may start at the insertion end proximate the first side of the tubular member and may extend back towards the second side of the tubular member. As an example, the angled cut may extend at an angle ranging from eight degrees to twelve degrees with respect to an outer surface of the tubular member proximal of the angled cut. In some cases, the angled cut may extend at an angle of ten degrees with respect to an outer surface of the tubular member proximal of the angled cut member.

In some cases, the minimum wall thickness of the tubular member may range from 0.15 millimeters (0.006 inches) to 0.25 millimeters (0.010 inches) and the maximum wall thickness of the tubular member may range from 0.30 millimeters (0.012 inches) to 0.41 millimeters (0.016 inches). In some cases, the minimum wall thickness may be 0.20 millimeters (0.008 inches) and the maximum wall thickness may be 0.36 millimeters (0.014 inches).

In some instances, a medical assembly may include an outer member defining a lumen having an inner diameter (ID) and an inner member having an outer diameter (OD), where the inner member extends within the lumen of the outer member. A removable coupler is adapted to be advanced over the inner member and extend into a space between the ID of the outer member and the OD of the inner member in order to secure the inner member relative to the outer member.

In some cases, the removable coupler may include a tubular member that extends from an insertion end to a handle end. An angled cut may extend back from the insertion end. In some cases, the tubular member may have a wall thickness that varies circumferentially from a minimum wall thickness along a first side of the tubular member to a maximum wall thickness along an opposing second side of the tubular member. In some cases, the angled cut may start at the insertion end proximate the first side of the tubular member and may extend back towards the second side of the tubular member. In some cases, the inner member may include a delivery device for an embolic coil and the outer member may include a sheath extending over the embolic coil.

In some instances, a removable coupler may be adapted to releasably secure an inner member to an outer member. The removable coupler is adapted to be removed prior to use. The removable coupler includes a tubular member extending from an insertion end to a handle end. The tubular member has a wall thickness that varies circumferentially from a minimum wall thickness along one side of the tubular member to a maximum wall thickness along an opposing side of the tubular member. The tubular member defines a tubular member lumen extending therethrough and adapted to accommodate the inner member therein. The removable coupler includes an angled cut extending back from the insertion end.

In some cases, the angled cut may extend at an angle ranging from eight degrees to twelve degrees with respect to an outer surface of the tubular member proximal of the angled cut. In some cases, the minimum wall thickness may range from 0.15 millimeters (0.006 inches) to 0.25 millimeters (0.010 inches) and the maximum wall thickness may range from 0.30 millimeters (0.012 inches) to 0.41 millimeters (0.016 inches). In some cases, the tubular member may define an outer diameter that ranges from 1.4 millimeters (0.055 inches) to 1.7 millimeters (0.065 inches) and an inner diameter that ranges from 0.90 millimeters (0.035 inches) to 1.0 millimeters (0.040 inches). In some cases, the tubular member may have a uniform composition from the insertion end to the handle end.

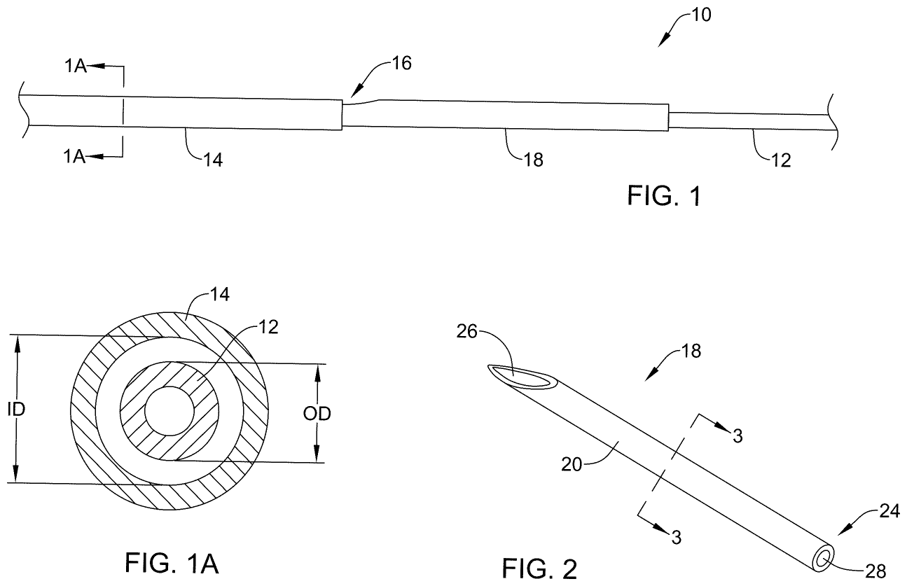

FIG. 1 is a schematic view of a medical assembly 10. The medical assembly 10 includes an inner member 12 and an outer member 14. The outer member 14 includes a lumen 16 that allows the inner member 12 to extend through the outer member 14. As an example, the inner member 12 may be a delivery wire for an implantable medical device such as an embolic coil. The outer member 14 may be a sheath that extends over the delivery wire and thus extends over the implantable medical device in order to hold the implantable medical device in position relative to the delivery wire. In some cases, the inner member 12 may represent any of a variety of different elongate medical devices that may be advanced through the outer member 14. The outer member 14 may represent a guide catheter, for example. The outer member 14 may represent an introducer sheath. In some cases, the outer member 14 may represent a removable sheath.

The medical assembly 10 includes a removable coupler 18 that may be used to selectively and/or temporarily secure the inner member 12 in position relative to the outer member 14. In some cases, as seen for example in FIG. 2, the removable coupler 18 may include a tubular member 20 that extends from an insertion end 22 to a handle end 24. The insertion end 22 may be adapted to be advanced into a space formed between an outer diameter (OD) of the inner member 12 and an inner diameter (ID) of the lumen 16 extending through the outer member 14. FIG. 1A is a cross-sectional view taken along the line 1A-1A of FIG. 1, labeling the OD of the inner member 12 and the ID of the outer member 14. The handle end 24 may be adapted to allow a user to grasp the removable coupler 18 by grasping the handle end 24. In some cases, the handle end 24 may have a length that is sufficient to allow a user to grasp the handle end 24 between two or more of their fingers, or one or more fingers and their thumb. As an example, the tubular member 20 may have an overall length of about 2.54 centimeters (one inch).

To secure the inner member 12 relative to the outer member 14, the removable coupler 18 may be advanced over a proximal end of the inner member 12 and may be slid down until the insertion end 22 extends into a space within the lumen 16, with the insertion end 22 extending between the OD of the inner member 12 and the ID of the outer member 14. When the user once again desires relative movement of the inner member 12 relative to the outer member 14, the user may simply withdraw the removable coupler 18 proximally from its position shown in FIG. 1. The user may completely withdraw the removable coupler 18 off the proximal end of the inner member 12. The user may also simply withdraw the removable coupler 18 any distance along the inner member 12 sufficient to permit desired movement of the inner member 12 relative to the outer member 14. In some cases, the removable coupler 18 may include a slit or perforation or otherwise weakened region within the tubular member 20 (not shown) that allows the removable coupler 18 to be split or peeled off the inner member 12.

The removable coupler 18 also includes an angled cut 26 and a lumen 28 extending through the tubular member 20 that allows the inner member 12 to extend through the removable coupler 18. The angled cut 26 starts at the insertion end 22 and extends proximally therefrom. In some cases, the tubular member 20 may have a uniform composition from its insertion end 22 to its handle end 24. In some cases, the tubular member 20 may be formed of two or more different materials. For example, the tubular member 20 may have a bilayer construction, with a first polymeric material forming an inner layer and a second polymeric material forming an outer layer. The inner layer may be formed of a lubricious material such that the tubular member 20 easily slides over the inner member 12 and the outer layer may be formed of a different material having a higher coefficient of friction such that the insertion end 22 has a higher frictional force when inserted between the OD of the inner member 12 and the ID of the outer member 14. In some cases, the tubular member 20 may include a first polymeric material forming the insertion end 22 and a second polymeric material forming the handle end 24. In some cases, when the tubular member 20 is formed of multiple polymers, or varying durometer versions of the same polymer, the tubular member 20 may be formed with visible markings that indicate where the angled cut 26 should be formed.

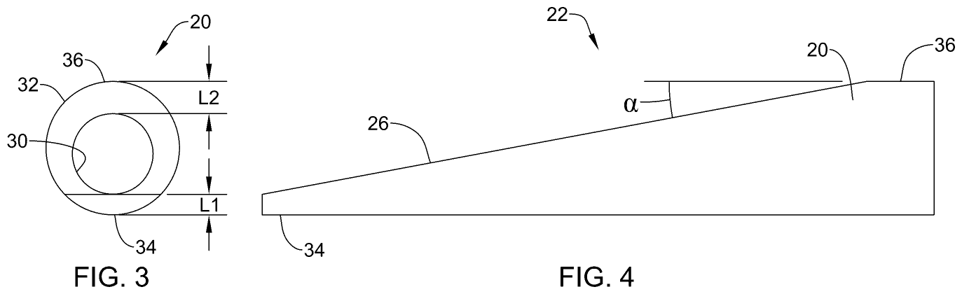

FIG. 3 is a cross-sectional view taken along the line 3-3 of FIG. 2, showing more details regarding an embodiment of the tubular member 20. In some cases, the tubular member 20 may have a uniform cross-sectional profile, with a uniform wall thickness circumferentially all the way around the tubular member 20. In some cases, as shown, the tubular member 20 may not have a uniform cross-sectional profile. Rather, the tubular member 20 may have a wall thickness (defined as a right-angle distance between an inner surface 30 and an outer surface 32 taken at any particular circumferential position) that varies circumferentially about the tubular member 20. As an example, the wall thickness may vary circumferentially from a minimum wall thickness labeled as L₁ along a first side 34 of the tubular member 20 to a maximum wall thickness labeled as L₂ along an opposing second side 36 of the tubular member 20. In some instances, the minimum wall thickness L₁ (measured at the first side 34) may range from 0.15 millimeters (0.006 inches) to 0.25 millimeters (0.010 inches) and the maximum wall thickness L₂ (measured at the second side 36) may range from 0.30 millimeters (0.012 inches) to 0.41 millimeters (0.016 inches). In an example, the minimum wall thickness L₁ may be 0.20 millimeters (0.008 inches) and the maximum wall thickness L₂ may be 0.36 millimeters (0.014 inches). In some cases, the tubular member 20 may have an outer diameter (proximal of the angled cut 26) that ranges from 1.4 millimeters (0.055 inches) to 1.7 millimeters (0.065 inches) and an inner diameter (proximal of the angled cut 26) that ranges from 0.90 millimeters (0.035 inches) to 1.0 millimeters (0.040 inches). It will be appreciated that these dimensions may vary, depending on the relative dimensions of the inner member 12 and the outer member 14. As an example, the dimensions provided herewith correspond to the inner member 12 having an OD in a range of 0.86 millimeters (0.034 inches) to 0.91 millimeters (0.036 inches) and the outer member 14 having an ID in a range of 1.1 millimeters (0.044 inches) to 1.2 millimeters (0.046 inches).

As shown, the inner surface 30 defines a first circular cross-sectional profile having a first diameter and the outer surface defines a second circular cross-sectional profile having a second diameter that is greater than the first diameter. The first circular cross-sectional profile may be considered as being radially offset from the second circular cross-sectional profile. This is achieved in a manufacturing process, for example, by offsetting the center of an extruder die from the center of an extruder tip.

FIG. 4 is an enlarged view of the insertion end 22 of the tubular member 20. The angled cut 26 may be formed at a controlled angle using a blade or a similar tool, for example. In some cases, the angled cut 26 extends from the first side 34 of the tubular member 20 (proximate where the wall thickness is at its minimum) to the second side 36 of the tubular member 20 (proximate where the wall thickness is at its maximum). The angled cut 26 may be considered as forming an angle alpha (α) with the outer surface of the tubular member 20. In some cases, the angle alpha (α) may be in a range of eight degrees to twelve degrees. In an example, the angle alpha (α) may be ten degrees. Forming the angled cut 26 extending from a minimum wall thickness to a maximum wall thickness can make it easier to insert the removable coupler 18 into the gap between the inner member 12 and the outer member 14. Having part of the tubular member 18 being thicker provides the user with a tactile feel of the wedge-lock because it can project over the outer diameter of the outer member 14. It can also be expected to be more forgiving, i.e., have acceptable performance, with dimensional variability of the inner member 12 and the outer member 14, and with variability in the insertion force.

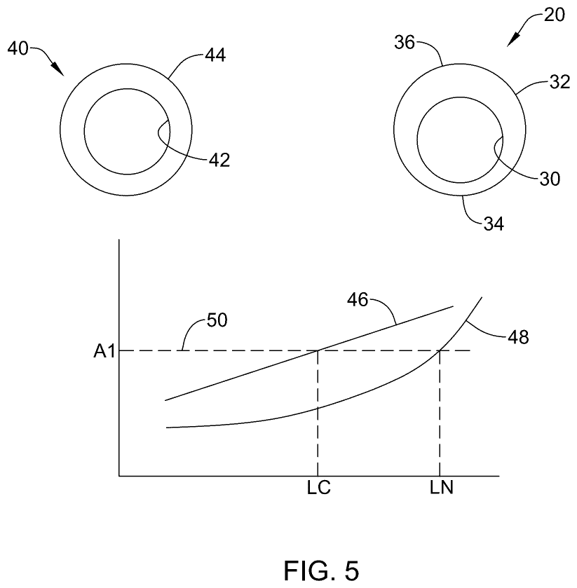

As noted, there are benefits to having the tubular member 20 having a non-concentric cross-sectional profile. FIG. 5 shows a concentric profile 40 in which an example tubular member has an inner surface 42 and an outer surface 44 with a constant perpendicular distance between the inner surface 42 and the outer surface 44. The concentric profile 40 has a constant wall thickness, regardless of circumferential position around the concentric profile 40. FIG. 5 also includes the non-concentric profile from FIG. 3. FIG. 5 includes a graphed line 46 that represents the area along the length of the angled cut 26 for a tubular member having a concentric profile such as the concentric profile 40. FIG. 5 also includes a graphed line 48 that represents the area along the length of the angled cut 26 for a tubular member having a non-concentric profile such as that repeated from FIG. 3. An Area A1, indicated by a dashed line 50 shows the area of the angled cut 26 that is sufficient to lock the inner member 12 and the outer member 14 together. The graphed line 46 intersects the dashed line 50 at a length LC while the graphed line 48 intersects the dashed line 50 at a length LN, which is greater than the length LC. Having a longer length of the angled cut 26 improves interface robustness. The concentric tube provides a smaller design window for the thickness of the tubular member 20 to balance out ease of insertion, and the force at which point the angled cut 26 will buckle.

The following data illustrates differences between having a concentric design and a non-concentric design. Table One shows insertion force and removal force for both a concentric design and a non-concentric design, with a 1.0 pound-force (lbf) insertion force. Table Two shows insertion force and removal force for both a concentric design and a non-concentric design, with a 1.5 lbf insertion force. Table Three shows insertion force and removal force for both a concentric design and a non-concentric design, with a 2.0 lbf insertion force. As can be seen in each case, the non-concentric wedge is easier to remove and has a lower variability in removal force.

TABLE One
Wedge Insertion Force of 4.44 Newtons (1.0 Pounds-Force)

| Concentric | | | | Non-Concentric | | | |
|---|---|---|---|---|---|---|---|
| Insertion Force | | Removal Force | | Insertion Force | | Removal Force | |
| Pounds- | | Pounds- | | Pounds- | | Pounds- | |
| Force | Newtons | Force | Newtons | Force | Newtons | Force | Newtons |
| 1 | 4.45 | 0.69 | 3.07 | 0.98 | 4.36 | 0.36 | 1.60 |
| 1 | 4.45 | 0.33 | 1.47 | 1.08 | 4.80 | 0.42 | 1.87 |
| 0.98 | 4.36 | 0.74 | 3.29 | 0.99 | 4.40 | 0.38 | 1.69 |
| 1.04 | 4.63 | 0.72 | 3.20 | 0.96 | 4.27 | 0.43 | 1.91 |
| 1 | 4.45 | 0.43 | 1.91 | 0.97 | 4.31 | 0.26 | 1.16 |

TABLE Two
Wedge Insertion Force of 6.67 Newtons (1.5 Pounds-Force)

| Concentric | | | | Non-Concentric | | | |
|---|---|---|---|---|---|---|---|
| Insertion Force | | Removal Force | | Insertion Force | | Removal Force | |
| Pounds- | | Pounds- | | Pounds- | | Pounds- | |
| Force | Newtons | Force | Newtons | Force | Newtons | Force | Newtons |
| 1.60 | 6.72 | 1.1 | 4.89 | 1.48 | 6.58 | 0.67 | 2.98 |
| 1.87 | 6.76 | 0.92 | 4.09 | 1.52 | 6.76 | 0.7 | 3.11 |
| 1.69 | 6.85 | 1.12 | 4.98 | 1.5 | 6.67 | 0.66 | 2.94 |
| 1.91 | 6.63 | 1.02 | 4.54 | 1.48 | 6.58 | 0.54 | 2.40 |
| 1.16 | 6.85 | 0.94 | 4.18 | 1.48 | 6.58 | 0.61 | 2.71 |

TABLE Three
Wedge Insertion Force of 8.90 Newtons (2.0 Pounds-Force)

| Concentric | | | | Non-Concentric | | | |
|---|---|---|---|---|---|---|---|
| Insertion Force | | Removal Force | | Insertion Force | | Removal Force | |
| Pounds- | | Pounds- | | Pounds- | | Pounds- | |
| Force | Newtons | Force | Newtons | Force | Newtons | Force | Newtons |
| 2.05 | 9.12 | 1.43 | 6.36 | 2.07 | 9.21 | 0.83 | 3.69 |
| 1.96 | 8.72 | 1.45 | 6.45 | 1.98 | 8.81 | 1 | 4.45 |
| 1.97 | 8.76 | 1.21 | 5.38 | 1.99 | 8.85 | 0.85 | 3.78 |
| 1.99 | 8.85 | 1.23 | 5.47 | 1.99 | 8.85 | 0.98 | 4.36 |
| 2 | 8.90 | 1.23 | 5.47 | 2.02 | 8.99 | 0.85 | 3.78 |

The tubular member 20 may be formed of a variety of thermoplastics such as various polyolefins and nylons, and stiffer polymers such as PEEK (polyetheretherketone) and PES (poly(ether-sulfone). The selected material should have stiffness and strength sufficient to withstand buckling during the insertion process. In some cases, various metals may also be used. There may be a tradeoff between the material used for the tubular member 20 and that used for the outer member 14. As an example, using a stiff material for the tubular member 20 may pair well with using a more pliable material for the outer member 14 in order to allow for some deformation as the removable coupler 18 is pushed in.

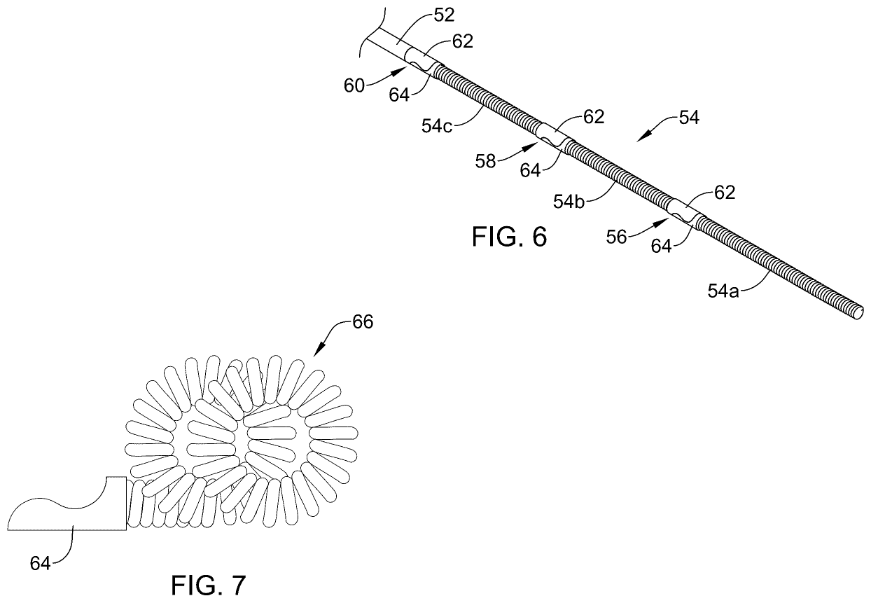

FIG. 6 is a perspective view of a delivery wire 52 that includes several embolic coils 54 that are coupled to the delivery wire 52. The embolic coils 54 are individually labeled as 54a, 54b and 54c. There may be more than three embolic coils 54, or there may be only one or two embolic coils 54. The delivery wire 52 may be considered as being an example of the inner member 12. The embolic coils 54 may be considered as being implantable medical devices that are coupled to the inner member 12. While not shown in FIG. 6, a removable sheath may be disposed over the delivery wire 52 in order to hold the embolic coils 54 in place relative to the delivery wire 52. The embolic coils 54 may be used to treat aneurysms for example, but other suitable occlusive medical devices transported, delivered, used, released etc. in a similar manner are also contemplated, including but not limited to stents, embolic filters, replacement heart valves, occlusion devices, and/or other medical implants, etc. In some cases, each of the embolic coils 54 may assume a first shape when coupled to the delivery wire 52 and may assume a second shape when disconnected from the delivery wire 52.

The embolic coil 54a is joined to the embolic coil 54b via a coupling 56. The embolic coil 54b is joined to the embolic coil 54c via a coupling 58. The embolic coil 54c is joined to the delivery wire 52 via a coupling 60. Each of the couplings 56, 58 and 60 include a first portion 62 that is part of one of the two elements being joined and a second portion 64 that is part of the other of the two elements being joined. When the first portion 62 and the second portion 64 are held together, such as via a sheath extending over the coupler 56, 58, 60, the first portion 62 and the second portion 64 are held together, and thus cannot separate. Removing the sheath with the exemplary described removable couplings 56, 58, 60 allows the first portion 62 and the second portion 64 to separate from each other. Thus, one or more embolic coils 54 may be released simply by controlling how far proximally an overlaying sheath (such as the outer member 14) is withdrawn.

FIG. 7 is a side view of the embolic coil 54a, showing the embolic coil 54a in a second configuration in which a coiled portion 66 has moved into a space-filling three dimensional shape now that the coiled portion 66 is no longer constrained. It will be appreciated that one or several embolic coils 54 may be implanted, as desired, in order to help fill a particular space. Additional details regarding the delivery wire 52 and the embolic coils 54 may be found in US 2022/0133329, which application is incorporated by reference.

The materials that can be used for the various components of the medical devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylenc (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical assembly, comprising:
an implantable medical device;
an elongate delivery member adapted to deliver the implantable medical device, the implantable medical device releasably secured to the elongate delivery member;
an outer sheath adapted to fit over the elongate delivery member; and
a removable coupler adapted to secure the elongate delivery member in position relative to the outer sheath when the removable coupler is disposed between the elongate delivery member and the outer sheath.

2. The medical assembly of claim 1, wherein the removable coupler comprises;
a tubular member extending from an insertion end to a handle end; and
an angled cut extending back from the insertion end.

3. The medical assembly of claim 2, wherein the tubular member has a wall thickness that varies circumferentially about the tubular member.

4. The medical assembly of claim 3, wherein the tubular member has a wall thickness that varies circumferentially from a minimum wall thickness along a first side of the tubular member to a maximum wall thickness along an opposing second side of the tubular member.

5. The medical assembly of claim 4, wherein the angled cut starts at the insertion end proximate the first side of the tubular member and extends back towards the second side of the tubular member.

6. The medical assembly of claim 5, wherein the angled cut extends at an angle ranging from eight degrees to twelve degrees with respect to an outer surface of the tubular member proximal of the angled cut.

7. The medical assembly of claim 5, wherein the angled cut extends at an angle of ten degrees with respect to an outer surface of the tubular member proximal of the angled cut.

8. The medical assembly of claim 4, wherein:
the minimum wall thickness ranges from 0.15 millimeters (0.006 inches) to 0.25 millimeters (0.010 inches); and
the maximum wall thickness ranges from 0.30 millimeters (0.012 inches) to 0.41 millimeters (0.016 inches).

9. The medical assembly of claim 4, wherein:
the minimum wall thickness is 0.20 millimeters (0.008 inches); and
the maximum wall thickness is 0.36 millimeters (0.014 inches).

10. The medical assembly of claim 1, wherein the implantable medical device comprises an embolic coil.

11. A medical assembly, comprising:
an outer member defining a lumen having an inner diameter (ID);
an inner member having an outer diameter (OD), the inner member extending within the lumen of the outer member; and
a removable coupler adapted to be advanced over the inner member and extend into a space between the ID of the outer member and the OD of the inner member in order to secure the inner member relative to the outer member.

12. The medical assembly of claim 11, wherein the removable coupler comprises;
a tubular member extending from an insertion end to a handle end; and
an angled cut extending back from the insertion end.

13. The medical assembly of claim 12, wherein the tubular member has a wall thickness that varies circumferentially from a minimum wall thickness along a first side of the tubular member to a maximum wall thickness along an opposing second side of the tubular member.

14. The medical assembly of claim 12, wherein the angled cut starts at the insertion end proximate the first side of the tubular member and extends back towards the second side of the tubular member.

15. The medical assembly of claim 11, wherein:
the inner member comprises a delivery device for an embolic coil; and
the outer member comprises a sheath extending over the embolic coil.

16. A removable coupler adapted to releasably secure an inner member to an outer member, the removable coupler adapted to be removed prior to use, the removable coupler comprising:
a tubular member extending from an insertion end to a handle end;
the tubular member having a wall thickness that varies circumferentially from a minimum wall thickness along one side of the tubular member to a maximum wall thickness along an opposing side of the tubular member;
the tubular member defining a tubular member lumen extending therethrough and adapted to accommodate the inner member therein; and
an angled cut extending back from the insertion end.

17. The removable coupler of claim 16, wherein the angled cut extends at an angle ranging from eight degrees to twelve degrees with respect to an outer surface of the tubular member proximal of the angled cut.

18. The removable coupler of claim 16, wherein:
the minimum wall thickness ranges from 0.15 millimeters (0.006 inches) to 0.25 millimeters (0.010 inches); and
the maximum wall thickness ranges from 0.30 millimeters (0.012 inches) to 0.41 millimeters (0.016 inches).

19. The removable coupler of claim 16, wherein:
the tubular member defines an outer diameter that ranges from 1.4 millimeters (0.055 inches) to 1.7 millimeters (0.065 inches); and
the tubular member defines an inner diameter that ranges from 0.90 millimeters (0.035 inches) to 1.0 millimeters (0.040 inches).

20. The removable coupler of claim 19, wherein the tubular member has a uniform composition from the insertion end to the handle end.
